(19) United States
(12) Patent Application Publication
(10) Pub. No.: US 2007/0015272 A1
(43) Pub. Date: Jan. 18, 2007

(54) Toxicity testing apparatus for cell stack cultures

(75) Inventors: Nobuhiko Ogura, Kanagawa-ken (JP); Yasuhiro Shimada, Minamiashigara-shi (JP); Satoru Toda, Minamiashigara-shi (JP); Nobufumi Mori, Kanagawa-ken (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD.

(21) Appl. No.: 11/485,274

(22) Filed: Jul. 13, 2006

(30) Foreign Application Priority Data
Jul. 13, 2005 (JP) 204367/2005

(51) Int. Cl. C12M 3/00

(52) U.S. Cl. 435/287.1

(57) ABSTRACT

A toxicity testing apparatus for testing toxicity of a sample with respect to a cell stack culture comprises an incubator for incubating at least one cell stack culture, a culture medium exchanger for exchanging a culture medium for the cell stack culture, a sample dispenser for adding the sample to the cell stack culture, and a detector for detecting an effect of the sample with respect to the cell stack culture.

### BACKGROUND OF THE INVENTION

1. Field of the Invention

This invention relates to a toxicity testing apparatus. This invention particularly relates to a toxicity testing apparatus for testing toxicity of a sample with respect to a cell stack culture.

2. Description of the Related Art

Toxicity tests using experimental animals have heretofore been performed for evaluating toxicity and irritant actions of products, which are to be brought into contact with organisms or to be administered to the organisms, such as pharmaceutical preparations, cosmetic preparations, and detergents, or chemical substances utilized for the production of the aforesaid products. However, considerable time and labor are required to perform animal experiments. Therefore, the toxicity tests using the experimental animals are not suitable for evaluations of a large number of samples. Also, recently, from the view point of protection of animals, there is a strong demand for in vitro toxicity testing methods, which will be capable of being employed as substitute methods for the animal experiments, and apparatuses or systems for carrying out the in vitro toxicity testing methods.

Ordinarily, in conventional in vitro toxicity testing methods, unicellular cultures or simple cultures of animal cells are utilized. However, ordinarily, survival periods of the unicellular cultures or the simple cultures of animal cells are as short as one week to two weeks. Therefore, with the conventional in vitro toxicity testing methods utilizing the unicellular cultures or the simple cultures of the animal cells, the problems are encountered in that long-term toxicity evaluation, such as the evaluation corresponding to subacute toxicity tests, which are performed over a period of approximately one month to approximately three months as in the cases of the animal experiments, is not capable of being made. Further, with the conventional in vitro toxicity testing methods utilizing the unicellular cultures or the simple cultures of the animal cells, the problems are encountered in that the evaluation is not capable of being made with respect to long-term phenomena, such as the toxicity of metabolic products occurring when the samples are metabolized by the cells.

Methods of testing the toxicity, the irritant actions, and the like, with respect to collagen gel embedded cultures, cultures on biopolymer matrixes, or tissue-wise cultures of animal cells, such as fibroblasts, fat cells, and vascular endothelial cells, have been reported in, for example, Japanese Unexamined Patent Publication No. 11(1999)-142392. However, each of the cultures utilized in the reported methods of testing the toxicity, the irritant actions, and the like, is the one obtained from processing, wherein the cells are, for example, embedded in the collagen gel and merely superposed in three-dimensional form. Therefore, the cultures utilized in the reported methods of testing the toxicity, the irritant actions, and the like, are not capable of being incubated for a long period of time. Also, with the reported methods of testing the toxicity, the irritant actions, and the like, wherein skin cells, the fat cells, and the like, are utilized, the toxicity of metabolic products is not capable of being evaluated.

Also, methods of controlling incubation of organism cells, incubating apparatuses for carrying out the methods of controlling incubation of organism cells, and methods of bringing active substances into contact with model cells and estimating effects of the active substances with respect to organisms have been reported in, for example, Japanese Unexamined Patent Publication No. 2003-235544 and U.S. Patent Application Publication Ser. No. 20050004765. However, in, for example, Japanese Unexamined Patent Publication No. 2003-235544 and U.S. Patent Application Publication Ser. No. 20050004765, nothing is described with respect to systems or apparatuses for incubating the cells for a long period of time and testing the toxicity of the samples.

Techniques for stacking cells by use of a cell culture carrier having a multi-layer structure, which contains a polymer hydrogel layer and a cell adhesive layer, such that the cells are capable of being incubated for a long period of time, have been proposed in, for example, U.S. Patent Application Publication Ser. No. 20030228693, U.S. Patent Application Publication Ser. No. 20050042745, and Japanese Unexamined Patent Publication No. 2005-110537.

The inventors have found that, in cases where a cell stack culture of the type as described in, for example, U.S. Patent Application Publication Ser. Nos. 20030228693, 20050042745, or Japanese Unexamined Patent Publication No. 2005-110537 is incubated and kept for a long period of time, and a sample is added to the cell stack culture, the toxicity of the sample and a metabolic product of the sample with respect to the cell stack culture is capable of being evaluated over a long period of time. However, the problems are encountered in that, since there is no apparatus or system capable of automatically performing the series of operations for the evaluation, a large number of samples are not capable of being evaluated or tested efficiently.

### SUMMARY OF THE INVENTION

The primary object of the present invention is to provide a toxicity testing apparatus, wherein toxicity of a sample with respect to a cell stack culture is capable of being tested automatically over a long period of time.

The present invention provides a toxicity testing apparatus for testing toxicity of a sample with respect to a cell stack culture, the apparatus comprising:

i) an incubator acting as incubation means for incubating at least one cell stack culture,

ii) a culture medium exchanger acting as culture medium exchanging means for exchanging a culture medium for the cell stack culture,

iii) a sample dispenser acting as sample adding means for adding the sample to the cell stack culture, and

iv) a detector acting as detection means for detecting an effect of the sample with respect to the cell stack culture.

With the constitution of the toxicity testing apparatus in accordance with the present invention, the cell stack cultures are capable of being incubated and kept automatically over a long period of time, and the toxicity test is capable of being efficiently performed in a simple manner and with respect to a plurality of samples.

The toxicity testing apparatus in accordance with the present invention should preferably be modified such that the sample adding means performs the addition of the sample with an addition quantity being set at various different values.

The term “addition of a sample with an addition quantity being set at various different values” as used herein means that the sample is added such that a concentration of the sample in the culture medium may be set at various different values. For example, in cases where titer plates are utilized as incubating vessels, a volume of the dispensed sample may be varied for different titer plates or for different wells of the titer plate. Alternatively, the sample having one of different concentrations may be prepared previously, and a predetermined quantity of the thus prepared sample may be added to the cell stack culture. In this manner, the addition quantity of the sample may be set at various different values. In cases where the evaluation using the samples having various different concentrations is made in a parallel manner, dependence on the concentration or the dependence on a dosage is capable of being specified efficiently.

Also, the toxicity testing apparatus in accordance with the present invention may be modified such that the sample adding means performs the addition of the sample a plurality of times at predetermined intervals and with a predetermined number of times. The term “performing addition of a sample a plurality of times at predetermined intervals and with a predetermined number of times” as used herein means that the addition of the sample to the cell stack culture is iterated the predetermined number of times and at the predetermined intervals. In cases where the addition of the sample to the cell stack culture is iterated, loss of the sample due to the culture medium exchanging operation, deactivation, and the like, is capable of being compensated for, and the long-term toxicity evaluation is capable of being made. Further, in such cases, an alteration of the toxicity in accordance with an addition schedule is capable of being specified.

Further, the toxicity testing apparatus in accordance with the present invention should preferably be modified such that the detection means is provided with a state detector acting as state detecting means for detecting a state of the cell stack culture and/or an assay device acting as assay means for assaying the culture medium after being used for the incubation of the cell stack culture. No limitation is imposed upon the type of the state detecting means. For example, the state detecting means may be means for detecting at least one item selected from the group consisting of a morphology of the cell stack culture and a number of surviving cells in the cell stack culture. Also, no limitation is imposed upon the type of the assay means. By way of example, the assay means may be means for assaying at least one item with respect to the culture medium after being used for the incubation of the cell stack culture, which at least one item is selected from the group consisting of an activity of GTP in the culture medium, an activity of γ-GOT in the culture medium, an activity of cytochrome P450 in the culture medium, a quantity of glucose in the culture medium, a quantity of albumin in the culture medium, a quantity of bile acid in the culture medium, a quantity of urea in the culture medium, a pH value of the culture medium, and a color of the culture medium. The term “culture medium after being used for an incubation of a cell stack culture” as used herein means the culture medium after being used for the incubation of the cell stack culture therein for a predetermined period of time. Ordinarily, the assay may be made by use of apart of the culture medium having been sampled from the incubating vessel. Alternatively, the assay may be made directly with respect to the culture medium in the state in which the culture medium is being contained in the incubating vessel.

Furthermore, the toxicity testing apparatus in accordance with the present invention should preferably be modified such that the apparatus further comprises a computer acting as analysis means for analyzing information representing results obtained from the detection means. The toxicity testing apparatus in accordance with the present invention should more preferably be modified such that the apparatus further comprises a data base acting as storage means for storing information representing results of the analysis obtained from the analysis means.

Also, the toxicity testing apparatus in accordance with the present invention should preferably be modified such that the cell stack culture contains:

(a) at least one cell layer containing hepatic parenchymal cells, and

(b) at least one cell layer containing cells selected from the group consisting of vascular endothelial cells, vascular smooth muscle cells, fat cells, blood corpuscle cells, and macrophages, and

the apparatus is adapted for testing toxicity of the sample to a liver.

In cases where the cell layer containing the hepatic parenchymal cells and the cell layer containing the vascular endothelial cells, or the like, are employed as the cell layers to be stacked, a three-dimensional tissue structure of the liver is capable of being constructed. Therefore, for example, the activity of the enzyme, such as GTP, γ-GOT, or cytochrome P450, which activity may act as an index for functions of the hepatic parenchymal cells, is capable of being assayed, and the toxicity of the sample to the liver, which is a drug-metabolizing organ, is capable of being evaluated.

The term “cell stack culture” as used herein means the stack of cell layers or cell sheets. The term “cell layer” as used herein means the cells incubated in the layer form on a cell incubating carrier, such as a laboratory dish or an extracellular matrix, e.g. collagen.

The term “cell sheet” as used herein means the sheet containing the cell layer and the cell incubating carrier. The cell layers or the cell sheets constituting the cell stack culture may be stacked such that the cells may be in contact with one another. Alternatively, the cell layers or the cell sheets constituting the cell stack culture may be stacked with the extracellular matrix intervening between the cell layers or the cell sheets. Also, no limitation is imposed upon the kinds of the cells contained in the cell layers or the cell sheets constituting the cell stack culture. Further, the kinds of the cells contained in the cell layers or the cell sheets constituting the cell stack culture maybe identical for the different layers or the different sheets. Alternatively, the kinds of the cells contained in the cell layers or the cell sheets constituting the cell stack culture may vary for the different layers or the different sheets. Furthermore, no limitation is imposed upon the number of the stacked cell layers or he stacked cell sheets.

With the toxicity testing apparatus in accordance with the resent invention, the toxicity of a plurality of samples with respect to the cell stack culture is capable of being tested automatically over a long period of time. Also, with the toxicity testing apparatus in accordance with the present invention, the evaluation is capable of being made with respect to long-term phenomena, such as the toxicity of metabolic products occurring when the samples are metabolized by the cells. Therefore, with the in vitro experiments, data having a high reliability is capable being obtained efficiently and in a simple manner with respect to the toxicity of the samples. Further, in cases where the acquired information representing the results of the analyses with respect to the plurality of the samples, or the like, is stored in the data base, it becomes possible to estimate the toxicity of a novel chemical substance, or the like, by use of the data base.

### BRIEF DESCRIPTION OF THE DRAWINGS

FIG. 1 is a block diagram showing an embodiment of the toxicity testing apparatus in accordance with the present invention, and

FIG. 2 is a conceptual view showing the embodiment of the toxicity testing apparatus in accordance with the present invention.

### DESCRIPTION OF THE PREFERRED EMBODIMENTS

The present invention will herein below be described in further detail with reference to the accompanying drawings.

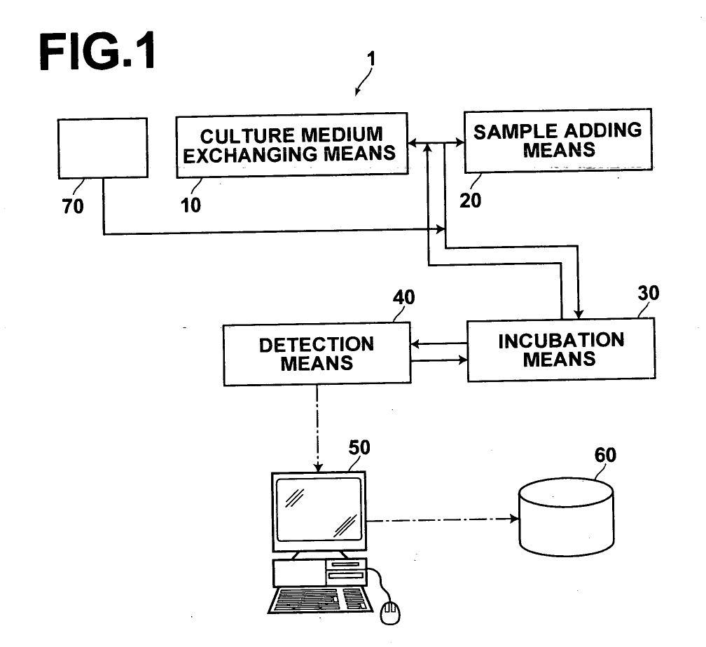

FIG. 1 is a block diagram showing an embodiment of the toxicity testing apparatus in accordance with the present invention.

With reference to FIG. 1, a toxicity testing apparatus 1, which is an embodiment of the toxicity testing apparatus in accordance with the present invention, comprises incubation means 30, culture medium exchanging means 10, sample adding means 20, detection means 40, analysis means 50, and storage means 60. When necessary, the toxicity testing apparatus 1 may also comprise cell setting means 70 for storing cell stack cultures before the cell stack cultures are to be used or for thawing frozen cell stack cultures, which have been stored, into the usable state. Further, though not shown, the toxicity testing apparatus 1 is provided with means for conveying incubating vessels, which contain the cell stack cultures, among the aforesaid various means. Furthermore, though not shown, the toxicity testing apparatus 1 is provided with control means for controlling the operations of the aforesaid various means.

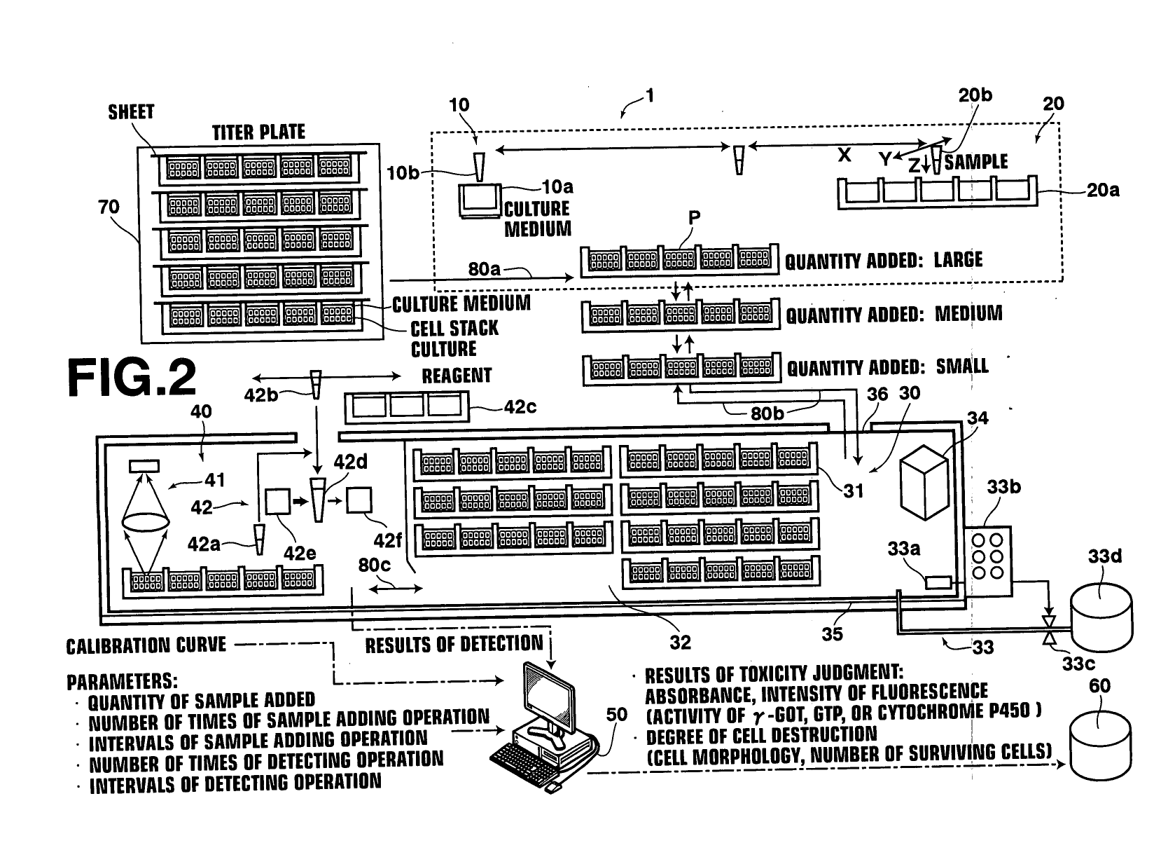

FIG. 2 is a conceptual view showing the embodiment of the toxicity testing apparatus in accordance with the present invention.

Firstly, testing steps performed by the toxicity testing apparatus 1 will be described herein below with reference to FIG. 2. In this embodiment, titer plates are utilized as the incubating vessels for the cell stack cultures.

Specifically, firstly, the titer plates containing the cell stack cultures are located in the cell setting means 70. At this time, in cases where the cell stack cultures having been frozen and stored at a temperature falling within the range of, for example, −40° C. to −80° C. have been located in the cell setting means 70, the cell stack cultures are thawed by the cell setting means 70 into the usable state. Thereafter, each of the titer plates is conveyed by conveyance means 80a to a plate support position P. The titer plate having thus been conveyed is supported at the plate support position P. At the plate support position P, a culture medium exchanging operation is performed by the culture medium exchanging means 10. Also, at the plate support position P, a sample adding operation is performed by the sample adding means 20.

After the sample adding operation has been performed by the sample adding means 20, the titer plate is conveyed by conveyance means 80b into the incubation means 30. In the incubation means 30, an incubating operation is performed for a predetermined period of time. When the predetermined period of time, e.g. one day, two days, or one week, has elapsed after the first sample adding operation was performed, the titer plate is conveyed by conveyance means 80c to the detection means 40. At the detection means 40, an image representing the state of the cell stack culture is recorded by state detecting means 41. Also, the culture medium is sampled by sampling means 42a from each of wells of the titer plate. The thus sampled culture medium is assayed by assay means 42. Thereafter, the titer plate is returned by the conveyance means 80c into the incubation means 30, and the incubation for the titer plate is continued.

During the toxicity test performed by the toxicity testing apparatus 1, the culture medium exchanging operation is performed periodically. Specifically, at predetermined intervals falling within the range of, for example, 0.5 day to two days, the titer plate is conveyed by the conveyance means 80b between the incubation means 30 and the plate support position P, and the culture medium is exchanged by the culture medium exchanging means 10. The incubation of the cell stack culture is performed for a period of time of, for example, at least two weeks, preferably for a period of time ranging from two weeks to three months.

The sample adding operation may be performed only one time. Alternatively, the sample adding operation maybe performed a plurality of times at desired intervals and with a predetermined number of times. For example, the sample adding operation may be performed at intervals of, for example, several days or one week, or at the time of each of the culture medium exchanging operations. Also, for example, the volume of the dispensed sample may be varied for different wells of the titer plate. Alternatively, the sample having one of different concentrations maybe prepared previously, and a predetermined quantity of the thus prepared sample may be added to the cell stack culture. In this manner, the addition quantity of the sample may be set at various different values. By way of example, the ultimate concentration of the sample in the culture medium may be varied for different wells of the titer plate and in stages by ½, by ⅓, by ⅕, or by 1/10 per stage.

In this embodiment, the cell stack culture is directly conveyed from the cell setting means 70 to the plate support position P, and the culture medium exchanging operation and the sample adding operation are performed. However, no limitation is imposed upon the period, at which the first culture medium exchanging operation and the first sample adding operation are performed, or the timing, with which the first culture medium exchanging operation and the first sample adding operation are performed. For example, alternatively, the cell stack culture may firstly be conveyed from the cell setting means 70 into the incubation means 30 and subjected to the incubating operation for a predetermined period of time, and thereafter the cell stack culture may be conveyed to the plate support position P and subjected to the culture medium exchanging operation and the sample adding operation. As another alternative, the cell stack culture may firstly be subjected to the culture medium exchanging operation, which is performed by the culture medium exchanging means 10, and may then be conveyed into the incubation means 30 and subjected to the incubating operation for a predetermined period of time, and the sample adding operation may thereafter be performed on the cell stack culture.

Each of the various means constituting the toxicity testing apparatus 1 will be described herein below.

The culture medium exchanging means 10 is provided with discharging means (not shown) for discharging the culture medium from the titer plate into a waste liquid tank. The culture medium exchanging means 10 is also provided with injection means 10b for injecting a new culture medium (e.g., aD-MEM culture medium, anMEM culture medium, an HamF12 culture medium, or an HamF10 culture medium) from a culture medium tank 10a into the titer plate. The culture medium exchanging means 10 is further provided with a support section (not shown) for supporting the discharging means and the injection means 10b. The discharging means sucks up the culture medium from the titer plate by use of a discharging pump (not shown) and via a sterilized tip, which has been releasably fitted to an end section. The discharging means discharges the thus sucked culture medium via a tube into the waste liquid tank. The injection means 10b sucks up a new culture medium from the culture medium tank 10a by use of an injecting pump (not shown) and via a tube and injects a predetermined quantity of the thus sucked new culture medium into the titer plate via a sterilized tip, which has been releasably fitted to an end section. The support section (not shown) supports the discharging means and the injection means 10b at independent sections. Alternatively, the support section (not shown) may support the discharging means and the injection means 10b together with each other. The support section (not shown) moves the discharging means and the injection means 10b and adjusts the positions of the discharging means and the injection means 10b with respect to the position of the titer plate.

The sample adding means 20 is provided with a dispensing pipette 20b for dispensing the sample. The sample adding means 20 is also provided with a support section (not shown) for supporting the dispensing pipette 20b. The support section (not shown) for supporting the dispensing pipette 20b is capable of moving in an X axis direction (i.e., in a direction parallel with the plane of the sheet of FIG. 2), a Y axis direction (i.e., in a direction normal to the plane of the sheet of FIG. 2), and in a Z axis direction (i.e., in a vertical direction in FIG. 2) . A sterilized tip is releasably fitted to an end section of the dispensing pipette 20b. Firstly, the dispensing pipette 20b is moved by the support section (not shown) of the sample adding means 20, and the position of the dispensing pipette 20b is matched with the position of a sample storing vessel 20a for storing the sample. A predetermined quantity of the sample is thus sucked up from the sample storing vessel 20a into the dispensing pipette 20b via the sterilized tip having been fitted to the end section. Thereafter, the dispensing pipette 20b is moved by the support section (not shown), and the position of the dispensing pipette 20b is matched with the position of the titer plate. The sample having been sucked into the dispensing pipette 20b is then introduced into the titer plate via the sterilized tip.

Also, though not shown, such that contamination may be prevented from occurring, an entire room, in which the culture medium exchanging means 10, the sample adding means 20, the culture medium tank 10a, the waste liquid tank (not shown), and the sample storing vessel 20a are accommodated, should preferably be cooled to a temperature falling within the range of 4° C. to 10° C. by use of, for example, an electronic heating and cooling system, such as a Peltier system. Alternatively, several means among the aforesaid various means may be cooled in the manner described above.

Further, in order for contamination to be prevented from occurring, a control operation should preferably be performed for exchanging the tips, which come into direct contact with the culture medium, the sample, and the like, with appropriate timing, for example, for each of the incubating vessels, for each of the samples, or at predetermined time intervals. Though not shown, a tip supply section, which accommodates a plurality of tips, and a tip recovering section, which recovers used tips, should preferably be located within the range of the movements of the culture medium exchanging means 10 and the sample adding means 20.

Furthermore, though not shown, in cases where a titer plate provided with a cover is utilized as the incubating vessel, the toxicity testing apparatus 1 may be provided with means for opening and closing the cover of the titer plate at the time of the culture medium exchanging operation or at the time of the sample adding operation.

In this embodiment, at the time of the culture medium exchanging operation, the titer plate is supported at the predetermined position (i.e., the plate support position P), the discharging means and the injection means 10b are moved by the corresponding support section, and the positions of the discharging means and the injection means 10b are matched with the position of the titer plate having been supported at the predetermined position. Also, at the time of the sample adding operation, the titer plate is supported at the predetermined position (i.e., the plate support position P), the dispensing pipette 20b is moved by the corresponding support section, and the position of the dispensing pipette 20b is matched with the position of the titer plate having been supported at the predetermined position. Alternatively, a moving mechanism (not shown), such as a conveyor, may be located between the culture medium exchanging means 10 and the sample adding means 20, the titer plate may be moved horizontally by the moving mechanism, and the position matching with respect to the horizontal direction may thereby be performed.

Also, in this embodiment, the culture medium exchanging means 10 and the sample adding means 20 are constituted as two independent means. Alternatively, for example, in cases where the discharging means and the injection means 10b constituting the culture medium exchanging means 10 are the ones utilizing a mechanism of the type identical with the mechanism of the dispensing pipette 20b of the sample adding means 20, the same common means may be utilized as both the culture medium exchanging means 19 and the sample adding means 20. For example, a support section may be constituted for movement among the waste liquid tank of the culture medium exchanging means 10, the culture medium tank 10a of the culture medium exchanging means 10, the sample storing vessel 20a of the sample adding means 20, and the plate support position P. In such cases, the culture medium exchanging operation containing the discharging of the culture medium from the titer plate into the waste liquid tank and the injection of a new culture medium from the culture medium tank 10a into the titer plate, and the sample adding operation, i.e., the dispensing of the sample from the sample storing means 20a into the titer plate, are capable of being performed by use of a single means, which has one pipette and one support section.

The incubation means 30 incubates and keeps the cell stack cultures. The incubation means 30 is provided with an accommodation chamber 32 capable of accommodating a plurality of titer plates 31, 31, . . . therein. The incubation means 30 is also provided with a CO₂ control section 33, a humidity control section 34, a temperature control section 35, and an opening section 36, which is capable of being opened and closed. Further, though not shown, the incubation means 30 should preferably be provided with a moving mechanism, which is capable of performing horizontal movements, vertical movements, and rotating movements of each of the titer plates 31, 31, . . . for conveying each of the titer plates 31, 31, . . . into the accommodation chamber 32 and out of the accommodation chamber 32 at the time of the culture medium exchanging operation, at the time of a detecting operation described later, and the like. The accommodation chamber 32 is provided with a plurality of vertically arrayed compartments, each of which is capable of accommodating at least one incubating vessel. Each of the compartments is kept in an unsealed state and under predetermined incubating conditions, such as the temperature, the humidity, and the CO₂concentration, within the accommodation chamber 32. The CO₂control section 33 is provided with a CO₂ sensor 33a, a CO₂ controller 33b, a pressure adjusting valve 33c, and a CO₂ supply source 33d. The CO₂ control section 33 controls the CO₂ concentration in the accommodation chamber 32 at a value falling within the range of approximately 5% to 10%, preferably at a value of 5%. The humidity control section 34 controls the humidity in the accommodation chamber 32 at a value falling within the range of 90% to 100%, preferably at a value of approximately 100%. The temperature control section 35 is constituted of a temperature control plate. The temperature control section 35 reliably controls the temperature in the accommodation chamber 32 at approximately 37° C. by use of the electronic heating and cooling system, such as the Peltier system. The accommodation chamber 32 is sealed from the exterior by the closing of the opening section 36 and keeps the cell stack cultures under the predetermined incubating conditions.

The detection means 40 is provided with state detecting means 41 for detecting the state of each of the cell stack cultures. The detection means 40 is also provided with assay means 42 for assaying the culture medium. No limitation is imposed upon the type of the state detecting means 41. By way of example, the state detecting means 41 may be constituted of a microscope provided with a CCD camera and may record an image representing the state of the cell stack culture contained in each of the wells of the titer plate. The assay means 42 is provided with sampling means 42a for sampling a predetermined quantity of the culture medium from each of the wells into an assay vessel 42d. The assay means 42 is also provided with reagent adding means 42b for dispensing a reagent, which corresponds to the measurement object, from a reagent vessel 42c into the assay vessel 42d. The assay means 42 is also provided with reagent adding means 42b for dispensing a reagent, which corresponds to the measurement object, from a reagent vessel 42c into the assay vessel 42d. The assay means 42 is further provided with a light source 42e for producing light having a desired wavelength. The assay means 42 is still further provided with a photo detector 42f for detecting fluorescence, which is produced by the resulting reaction product when the light produced by the light source 42e is irradiated to the reaction product, the light, which has passed through the reaction product when the light produced by the light source 42e is irradiated to the reaction product, or the light, which has been reflected from the reaction product when the light produced by the light source 42e is irradiated to the reaction product. In cases where the kind of the reagent added, the wavelength of the light produced by the light source 42e, and the type of the photo detector 42f are selected appropriately, assays are capable of being made with respect to various enzymes and organism substances, which are capable of acting as indexes for the cell functions, such as an activity of GTP in the culture medium, an activity of γ-GOT in the culture medium, an activity of cytochrome P450 in the culture medium, a quantity of glucose in the culture medium, a quantity of albumin in the culture medium, a quantity of bile acid in the culture medium, and a quantity of urea in the culture medium. Also, though not shown, the assay means 42 may be provided with a filter. In such cases, an assay is capable of being made with respect to a color of the culture medium. Further, though not shown, the assay means 42 may be provided with a pH meter. In such cases, an assay is capable of being made with respect to a pH value of the culture medium. As the reagent to be added, it is possible to use one of various fluorescent reagents, labeled reagents, and unlabeled reagents, which are available commercially.

The conveyance means 80a conveys the titer plate, which contains the cell stack cultures, between the cell setting means 70 and the plate support position P. The conveyance means 80b conveys the titer plate, which contains the cell stack cultures, between the plate support position P and the incubation means 30. Also, the conveyance means 80c conveys the titer plate, which contains the cell stack cultures, between the incubation means 30 and the detection means 40. No limitation is imposed upon the type of each of the conveyance means 80a, 80b, and 80c. By way of example, each of the conveyance means 80a, 80b, and 80c may be constituted of a conveyor, a conveyance robot having an arm section, a turn table, or the like.

The control means (not shown) is provided with a managing computer and controls the operations of the aforesaid various means in accordance with control signals given by the managing computer. Also, the control means may be provided with reading means, such as a bar code reader, for reading label data, such as bar code labels attached to the titer plates, the sample storing vessel 20a, and the like. The control means may send the thus read label data to the managing computer. The managing computer may be incorporated in the toxicity testing apparatus 1. Alternatively, the managing computer may be located at the exterior of the toxicity testing apparatus 1 and may be electrically connected to the control means of the toxicity testing apparatus 1.

The analysis means 50 contains a computer and an analysis program. The analysis means 50 receives the information representing the results of the detection, which information is sent from the detection means 40. The analysis means 50 also receives information representing calibration curves and information representing test parameters (e.g., the quantity of the sample added, the concentration of the sample, the number of times of the sample adding operation, the intervals of the sample adding operation, the number of times of the detecting operation, and the intervals of the detecting operation), and the like. The analysis means 50 makes the analysis and furnishes useful and detailed data in accordance with the results of the analysis. For example, the analysis means 50 calculates the concentration or the activity of the assay object substance in the culture medium from the information representing an absorbance or the intensity of the fluorescence, which information has been received from the assay means 42, and from the information representing the calibration curve. Also, the analysis means 50 analyzes the image data representing the cell stack culture, which image data has been received from the state detecting means 41. The analysis means 50 thus calculates a degree of cell destruction from the number of surviving cells and the cell morphology. Further, the analysis means 50 makes a comparison between the results of the detection or the results of the analysis obtained in cases where the sample was not added and in cases where the sample was added. In accordance with the results of the comparison, the analysis means 50 makes a judgment as to the toxicity of the sample. The managing computer of the control means described above may also acts as the computer of the analysis means 50.

The storage means 60 stores the information representing the results of the analysis such that it may be clear which results of the analysis correspond to which parameters (such as a chemical structure of the sample, the quantity of the sample added, the number of times of the sample adding operation, and the intervals of the sample adding operation). Also, though not shown, the information representing the results of the detection, which information is obtained from the detection means 40, may be directly sent to the storage means 60 and stored in the storage means 60.

The temperature in the cell setting means 70 is controlled by the electronic heating and cooling system, such as the Peltier system. The cell setting means 70 stores the cell stack cultures before the cell stack cultures are to be used. Also, in cases where the cell stack cultures having been frozen and stored at a temperature falling within the range of, for example, −40° C. to −80° C. have been located in the cell setting means 70, the cell stack cultures are thawed by the cell setting means 70 into the usable state.

The cell stack culture employed in the toxicity testing apparatus in accordance with the present invention is the stack of the cell layers, which are constituted of the cells incubated in the layer form on a cell incubating carrier, or the stack of the cell sheets, each of which contains the cell layer and the cell incubating carrier. The cell stack culture enables long-term incubation to be performed. By way of example, the cell stack culture may be prepared by use of a cell culture carrier having a multi-layer structure, which contains a polymer hydrogel layer and a cell adhesive layer, as described in, for example, U.S. Patent Application Publication Ser. Nos. 20030228693, 20050042745, and Japanese Unexamined Patent Publication 2005-110537. The cell layers or the cell sheets constituting the cell stack culture may be stacked such that the cells may be in contact with one another. Alternatively, the cell layers or the cell sheets constituting the cell stack culture may be stacked with the extracellular matrix intervening between the cell layers or the cell sheets. Also, no limitation is imposed upon the kinds of the cells contained in the cell layers or the cell sheets constituting the cell stack culture. For example, the cells contained in the cell layers or the cell sheets constituting the cell stack culture may contain at least one kind of the cells selected from the group consisting of fibroblasts, vascular endothelial cells, chondrocytes, hepatic parenchymal cells, enterocytes, epidermal keratinocytes, osteoblasts, and bone marrow mesenchymal cells. Further, no limitation is imposed upon the number of the stacked cell layers or the stacked cell sheets. However, the number of the stacked cell layers or the stacked cell sheets should preferably fall within the range of two to ten, and should more preferably fall within the range of two to five. The kind of the culture medium, the incubating conditions, and the like, may be selected appropriately in accordance with the kind of the cells contained in the cell stack culture. Furthermore, no limitation is imposed upon the kind of the culture medium. For example, as the culture medium, it is possible to employ a D-MEM culture medium, an MEM culture medium, an HamF12 culture medium, or an HamF10 culture medium. Also, no limitation is imposed upon the type of the incubating vessel. For example, as the incubating vessel, it is possible to employ a 96-hole titer plate or a different type of an incubating plate.

Also, the toxicity testing apparatus in accordance with the present invention should preferably be modified such that the cell stack culture contains (a) at least one cell layer containing hepatic parenchymal cells, and (b) at least one cell layer containing cells selected from the group consisting of vascular endothelial cells, vascular smooth muscle cells, fat cells, blood corpuscle cells, and macrophages. In cases where the cell layer containing the hepatic parenchymal cells and the cell layer containing the vascular endothelial cells, or the like, are employed as the cell layers to be stacked, a three-dimensional tissue structure of the liver is capable of being constructed. Therefore, for example, the activity of the enzyme, such as GTP, γ-GOT, or cytochrome P450 in the culture medium, which activity may act as an index for the functions of the hepatic parenchymal cells, in particular the activity of the cytochrome P450, is capable of being assayed, and the toxicity of the sample to the liver, which is a drug-metabolizing organ, is capable of being tested. With the toxicity testing apparatus in accordance with the present invention, in cases where the cell stack culture described above is employed, the cell stack culture is capable of being incubated and kept over a period of time of as long as at least two weeks, and the toxicity of the sample is capable of being tested. Also, in such cases, besides the toxicity of the sample, the toxicity of the metabolic product of the sample is capable of being evaluated.

No limitation is imposed upon the kind of the sample to be tested with the toxicity testing apparatus in accordance with the present invention. For example, the samples may be novel chemical substances, chemical agents, pharmaceutical preparations, cosmetic preparations, or detergents. Also, the samples may be synthetic compounds, natural compounds, or compounds prepared through genetic engineering.

What is claimed is:

1. A toxicity testing apparatus for testing toxicity of a sample with respect to a cell stack culture, the apparatus comprising:
i) an incubator for incubating at least one cell stack culture,
ii) a culture medium exchanger for exchanging a culture medium for the cell stack culture,
iii) a sample dispenser for adding the sample to the cell stack culture, and
iv) a detector for detecting an effect of the sample with respect to the cell stack culture.

2. An apparatus as defined in claim 1 wherein the sample dispenser performs the addition of the sample with an addition quantity being set at various different values.

3. An apparatus as defined in claim 1 wherein the sample dispenser performs the addition of the sample a plurality of times at predetermined intervals and with a predetermined number of times.

4. An apparatus as defined in claim 1 wherein the detector is provided with a state detector for detecting a state of the cell stack culture.

5. An apparatus as defined in claim 4 wherein the state detector detects at least one item selected from the group consisting of a morphology of the cell stack culture and a number of surviving cells in the cell stack culture.

6. An apparatus as defined in claim 1 wherein the detector is provided with an assay device for assaying the culture medium after being used for the incubation of the cell stack culture.

7. An apparatus as defined in claim 6 wherein the assay device assays at least one item with respect to the culture medium after being used for the incubation of the cell stack culture, which at least one item is selected from the group consisting of an activity of GTP in the culture medium, an activity of γ-GOT in the culture medium, an activity of cytochrome P450 in the culture medium, a quantity of glucose in the culture medium, a quantity of albumin in the culture medium, a quantity of bile acid in the culture medium, a quantity of urea in the culture medium, a pH value of the culture medium, and a color of the culture medium.

8. An apparatus as defined in claim 1 wherein the apparatus further comprises a computer for analyzing information representing results obtained from the detector.

9. An apparatus as defined in claim 8 wherein the apparatus further comprises a data base for storing information representing results of the analysis obtained from the computer.

10. An apparatus as defined in claim 1 wherein the cell stack culture contains:
(a) at least one cell layer containing hepatic parenchymal cells, and
(b) at least one cell layer containing cells selected from the group consisting of vascular endothelial cells, vascular smooth muscle cells, fat cells, blood corpuscle cells, and macrophages, and
the apparatus is adapted for testing toxicity of the sample to a liver.

11. An apparatus as defined in claim 10 wherein the sample dispenser performs the addition of the sample with an addition quantity being set at various different values.

12. An apparatus as defined in claim 10 wherein the sample dispenser performs the addition of the sample a plurality of times at predetermined intervals and with a predetermined number of times.

13. An apparatus as defined in claim 10 wherein the detector is provided with a state detector for detecting a state of the cell stack culture.

14. An apparatus as defined in claim 13 wherein the state detector detects at least one item selected from the group consisting of a morphology of the cell stack culture and a number of surviving cells in the cell stack culture.

15. An apparatus as defined in claim 10 wherein the detector is provided with an assay device for assaying the culture medium after being used for the incubation of the cell stack culture.

16. An apparatus as defined in claim 15 wherein the assay device assays at least one item with respect to the culture medium after being used for the incubation of the cell stack culture, which at least one item is selected from the group consisting of an activity of GTP in the culture medium, an activity of γ-GOT in the culture medium, an activity of cytochrome P450 in the culture medium, a quantity of glucose in the culture medium, a quantity of albumin in the culture medium, a quantity of bile acid in the culture medium, a quantity of urea in the culture medium, a pH value of the culture medium, and a color of the culture medium.

17. An apparatus as defined in claim 10 wherein the apparatus further comprises a computer for analyzing information representing results obtained from the detector.

18. An apparatus as defined in claim 17 wherein the apparatus further comprises a data base for storing information representing results of the analysis obtained from the computer.

19. A toxicity testing apparatus for testing toxicity of a sample with respect to a cell stack culture, the apparatus comprising:
i) incubation means for incubating at least one cell stack culture,
ii) culture medium exchanging means for exchanging a culture medium for the cell stack culture,
iii) sample adding means for adding the sample to the cell stack culture, and
iv) detection means for detecting an effect of the sample with respect to the cell stack culture.
